(19) United States
(12) Patent Application Publication
(10) Pub. No.: US 2008/0103406 A1
(43) Pub. Date: May 1, 2008

(54) INTEGRATED SYSTEM FOR MANAGING PATIENTS WITH HEART FAILURE

(76) Inventor: Nader Kameli, Hugo, MN (US)

Correspondence Address: SCHWEGMAN, LUNDBERG & WOESSNER, P.A., P.O. BOX 2938, MINNEAPOLIS, MN, 55402, US

(21) Appl. No.: 11/553,103

(22) Filed: Oct. 26, 2006

(51) Int. Cl. A61B 5/02

(52) U.S. Cl. 600/528; 607/32

(57) ABSTRACT

An external heart sound sensor externally detects at least one heart sound from a patient, which information is used to automatically adjust one or more cardiac resynchronization therapy (CRT) or other control parameters of an implantable medical device, such as an implantable cardiac rhythm management device. An external telemetry circuit is coupled to the external heart sound sensor, and the telemetry circuit receives information about the at least one heart sound. The external telemetry circuit is also adapted to communicate with an implantable medical device for automatically programming at least one parameter of the implantable medical device using information about the at least one heart sound received from the external heart sound sensor.

### TECHNICAL FIELD

This document generally relates to cardiac rhythm management (CRM) systems and particularly to a system for externally sensing heart sounds and automatically programming an implantable medical device as a function of the sensed heart sounds.

### BACKGROUND

The heart is at the center of the circulatory system. It consists of four chambers—two atria and two ventricles. The right atrium receives deoxygenated blood from the body, pumps it into the right ventricle, and the right ventricle pumps the blood to the lungs to be re-oxygenated. The re-oxygenated blood returns to the left atrium, it is pumped into the left ventricle, and then the blood is pumped by the left ventricle throughout the body to meet the hemodynamic needs of the body.

Heart sounds are associated with mechanical vibrations from activity of a patient's heart and the flow of blood through the heart. Heart sounds recur with each cardiac cycle and are separated and classified according to the activity associated with the vibration. The first heart sound (S1) is the vibrational sound made by the heart during tensing of the mitral valve. The second heart sound (S2) marks the beginning of diastole. The third heart sound (S3) and fourth heart sound (S4) are related to filling pressures of the left ventricle during diastole. Heart sounds are useful indications of proper or improper functioning of a patient's heart.

Implantable medical devices (IMDs) are devices designed to be implanted into a patient. Some examples of these devices include cardiac function management (CFM) devices such as implantable pacemakers, implantable cardioverter defibrillators (ICDs), cardiac resynchronization devices, and devices that include a combination of such capabilities. The devices are typically used to treat patients using electrical therapy and to aid a physician or caregiver in patient diagnosis through internal monitoring of a patient's condition. The devices may include electrodes in communication with sense amplifiers to monitor electrical heart activity within a patient, and often include sensors to monitor other internal patient parameters. Other examples of implantable medical devices include implantable diagnostic devices, implantable insulin pumps, devices implanted to administer drugs to a patient, or implantable devices with neural stimulation capability.

### OVERVIEW

In an embodiment, an external heart sound sensor is adapted to externally detect at least one heart sound from a patient. An external telemetry circuit is coupled to the external heart sound sensor, and the telemetry circuit receives information about the at least one heart sound. The external telemetry circuit is also adapted to communicate with an implantable medical device for automatically programming at least one parameter of the implantable medical device using information about the at least one heart sound received from the external heart sound sensor.

In Example 1, a system includes an external heart sound sensor adapted to externally detect at least one heart sound from a patient. The system also includes an external telemetry circuit that is coupled to the external heart sound sensor to receive information about the at least one heart sound. The external telemetry circuit is further adapted to communicate with an implantable medical device for automatically programming at least one parameter of the implantable medical device using information about the at least one heart sound received from the external heart sound sensor.

In Example 2, the system of Example 1 optionally includes an external processor that is coupled to each of the external heart sound sensor and the external telemetry circuit. The external processor is optionally configured to automatically determine a value of the at least one parameter of the implantable medical device using the information about the at least one heart sound received from the external heart sound sensor. The system of Example 1 further optionally includes a user interface to obtain user-confirmation of the value of the at least one parameter in conjunction with the automatic programming of the at least one parameter.

In Example 3, in the systems of Examples 1-2, the external processor and the external telemetry circuit are optionally included within an external programmer for an implantable cardiac function management device, the external heart sound sensor is optionally associated with an external heart sound system that is housed separately from the external programmer, and the external heart sound system and the external programmer are optionally adapted to be communicatively intercoupled.

In Example 4, in the systems of Examples 1-3, at least a portion of the external processor, the external telemetry circuit, and the external heart sound sensor are all optionally included within an external programmer for an implantable cardiac function management device.

In Example 5, the systems of Examples 1-4 optionally include an implantable or external cardiac signal sensor and an external display. The external display is optionally coupled to the external heart sound sensor and the implantable or external cardiac signal sensor, and the external display is optionally configured to display a heart sound signal and a cardiac signal. Additionally, the external telemetry circuit is optionally adapted to communicate with the implantable medical device for automatically adjusting a cardiac resynchronization therapy parameter to decrease or minimize an observed heart sound amplitude received from the external heart sound sensor, and the observed heart sound amplitude optionally includes an observed S3 heart sound amplitude received from the external heart sound sensor.

In Example 6, the systems of Examples 1-5 optionally include the implantable medical device, and the external telemetry circuit is optionally adapted to communicate with an implantable medical device for automatically adjusting at least one of an atrioventricular (AV) delay, an interventricular (VV) delay, an LV offset, an intraventricular delay, and a selected electrode for delivering an electrostimulation pulse, and the implantable medical device optionally includes a cardiac resynchronization therapy circuit.

In Example 7, the systems of Examples 1-6 optionally include a remote device and an external network communication circuit adapted to communicate with the remote device using a computer or telecommunications network. The remote device optionally includes a centralized repository for data received from multiple implantable medical devices.

In example 8, the systems of Examples 1-7 optionally include one or more of a serial port, parallel port, and a wireless port, coupled between the external heart sound sensor and the external telemetry circuit. The serial port optionally includes an RS-232 port, a Universal Serial Bus (USB) port, or a fly-by-wire port.

In Example 9, a method includes externally detecting at least one heart sound signal from a patient, and communicating with an implantable medical device, the communicating including automatically programming at least one parameter of the implantable medical device using information about the at least one heart sound.

In Example 10, the method of Example 9 optionally includes automatically determining a value of the at least one parameter of the implantable medical device using the information about the at least one heart sound received from the external heart sound sensor, obtaining user-confirmation of the value of the at least one parameter in conjunction with the automatic programming of the at least one parameter, and detecting a cardiac signal from the patient, and displaying the heart sound signal and the cardiac signal on an external display unit.

In Example 11, the methods of Examples 9-10 optionally include automatically adjusting a cardiac resynchronization therapy parameter to decrease or minimize an externally detected heart sound amplitude. The externally detected heart sound amplitude optionally includes an externally detected S3 heart sound amplitude.

In Example 12, the methods of Examples 9-11 optionally include automatically communicating with the implantable medical device to automatically adjust at least one of an atrioventricular (AV) delay, an interventricular (VV) delay, an LV offset, an intraventricular delay, and a selected electrode for delivering an electrostimulation pulse.

In Example 13, the methods of Examples 9-12 optionally include communicating data from multiple implantable medical devices to a remote device using a computer or telecommunications network.

In Example 14 a system includes an external heart sound monitor that includes a terminal configured to receive at least one heart sound signal from an external heart sound sensor, and a port configured to communicate with an implantable medical device for automatically programming at least one parameter of the implantable medical device using information about the at least one heart sound received from the external heart sound sensor.

In Example 15, the system of Example 14 optionally includes a display unit to display the at least one heart sound signal received from the external heart sound sensor, and an external or implantable cardiac signal sensor coupled to the display, the external display is optionally configured to display a heart sound signal and a cardiac signal.

In Example 16, the systems of Examples 14-15 optionally include an external telemetry circuit that is coupled to the port. The external telemetry circuit is optionally adapted to communicate with the implantable medical device for the automatic programming of the at least one parameter of the implantable medical device using information about the at least one heart sound received from the external heart sound sensor. The systems of Examples 14-15 further optionally include an external processor that is coupled to each of the terminal and the port. The external processor is optionally configured to automatically determine a value of the at least one parameter of the implantable medical device using the information about the at least one heart sound received from the external heart sound sensor. The external telemetry circuit is optionally adapted to communicate with the implantable medical device for automatically adjusting at least one of an atrioventricular (AV) delay, an interventircular (VV) delay, an LV offset, an intraventricular delay, and a selected electrode for delivering an electrostimulation pulse. The external processor and the external telemetry circuit are optionally included within an external programmer for an implantable cardiac function management device. The external heart sound monitor is optionally associated with an external heart sound system that is housed separately from the external programmer. The heart sound system and the external programmer are optionally adapted to be communicatively intercoupled. Additionally, at least a portion of the external processor, the external telemetry circuit, and the external heart sound sensor are all optionally included within an external programmer for an implantable cardiac function management device.

In Example 17, in the systems of Examples 14-16, the external telemetry circuit is optionally adapted to communicate with the implantable medical device for automatically adjusting a cardiac resynchronization therapy parameter to decrease or minimize an observed heart sound amplitude received from the external heart sound sensor. The external telemetry circuit is optionally adapted to communicate with the implantable medical device for automatically adjusting a cardiac resynchronization therapy parameter to decrease or minimize an observed S3 heart sound amplitude from the external heart sound sensor.

In Example 18, the systems of Examples 14-17 optionally include the implantable medical device, and the implantable medical device optionally includes a cardiac resynchronization therapy circuit.

In Example 19, the systems of Examples 14-18 optionally include a remote device and an external network communication circuit optionally adapted to communicate with the remote device using a computer or telecommunications network. The remote device optionally includes a central repository for data received from multiple implantable medical devices.

In Example 20, the systems of Examples 14-19 optionally include a user interface to obtain user-confirmation of the value of the at least one parameter in conjunction with the automatic programming of the at least one parameter. The port optionally includes a serial RS-232 port, a Universal Serial Bus (USB) port, or a fly-by-wire port.

In Example 21, a system includes an external programmer including a port configured to receive at least one heart sound from an external heart sound sensor and an external telemetry circuit. The external telemetry circuit is coupled to the external programmer, and the external telemetry circuit adapted to communicate with an implantable medical device for automatically programming at least one parameter of the implantable medical device using information about the at least one heart sound received from the external programmer.

In Example 22, the systems of Examples 20-21 optionally include an external processor, coupled to each of the external programmer and the external telemetry circuit. The external processor is optionally configured to automatically determine a value of the at least one parameter of the implantable medical device using the information about the at least one heart sound received from the external programmer. In Example 22, the systems of Examples 20-21 further optionally include a user interface to obtain user-confirmation of the value of the at least one parameter in conjunction with the automatic programming of the at least one parameter. The external processor and the external telemetry circuit are optionally included within the external programmer, and the external heart sound sensor is optionally associated with an external heart sound system that is housed separately from the external programmer. The external heart sound system and the external programmer are optionally adapted to be communicatively intercoupled. At least a portion of the external processor, the external telemetry circuit, and the external heart sound sensor are all optionally included within the external programmer.

In Example 23, the systems of Examples 20-22 optionally include an implantable or external cardiac signal sensor and an external display. The external display is optionally coupled to the external heart sound sensor and the implantable or external cardiac signal sensor, and the external display is optionally configured to display a heart sound signal and a cardiac signal. The external telemetry circuit is optionally adapted to communicate with the implantable medical device for automatically adjusting a cardiac resynchronization therapy parameter to decrease or minimize an observed heart sound amplitude received from the external programmer. The external telemetry circuit is optionally adapted to communicate with the implantable medical device for automatically adjusting a cardiac resynchronization therapy parameter to decrease or minimize an observed S3 heart sound amplitude received from the external programmer.

In Example 24, the systems of Examples 20-22 optionally include a remote device, an external network communication circuit that is optionally adapted to communicate with the remote device using a computer or telecommunications network, and the implantable medical device. The external telemetry circuit is optionally adapted to communicate with an implantable medical device for automatically adjusting at least one of an atrioventricular (AV) delay, an interventricular (VV) delay, an LV offset, an intraventricular delay, and a selected electrode for delivering an electrostimulation pulse. The implantable medical device optionally includes a cardiac resynchronization therapy circuit. The remote device optionally includes a centralized repository for data received from multiple implantable medical devices.

In Example 25, the systems of Examples 20-24 optionally include a serial port coupled between the external heart sound sensor and the external telemetry circuit. The serial port optionally includes an RS-232 port, a Universal Serial Bus (USB) port, or a fly-by-wire port.

This overview relates to some of the teachings of the present application and it is not intended to be an exclusive or exhaustive treatment of the present subject matter. Further details about the present subject matter are found in the detailed description and appended claims. Other aspects of the invention will be apparent to persons skilled in the art upon reading and understanding the following detailed description and viewing the drawings that form a part thereof. The scope of the present invention is defined by the appended claims and their legal equivalents.

### BRIEF DESCRIPTION OF THE DRAWINGS

The drawings illustrate generally, by way of example, various embodiments discussed in the present document. The drawings are for illustrative purposes only and may not be to scale.

FIGS. 1 and 1A are illustrations of embodiments of a cardiac rhythm management (CRM) system and portions of the environment in which the CRM system operates.

FIGS. 2, 2A, 2B, and 2C are illustrations of other embodiments of a CRM system and portions of the environment in which the CRM system operates.

FIGS. 3, 3A, 3B, and 3C are illustrations of other embodiments of a CRM system and portions of the environment in which the CRM system operates.

FIG. 4 is an example of a process to externally detect heart sounds and program an implantable medical device as a function of the heart sounds.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

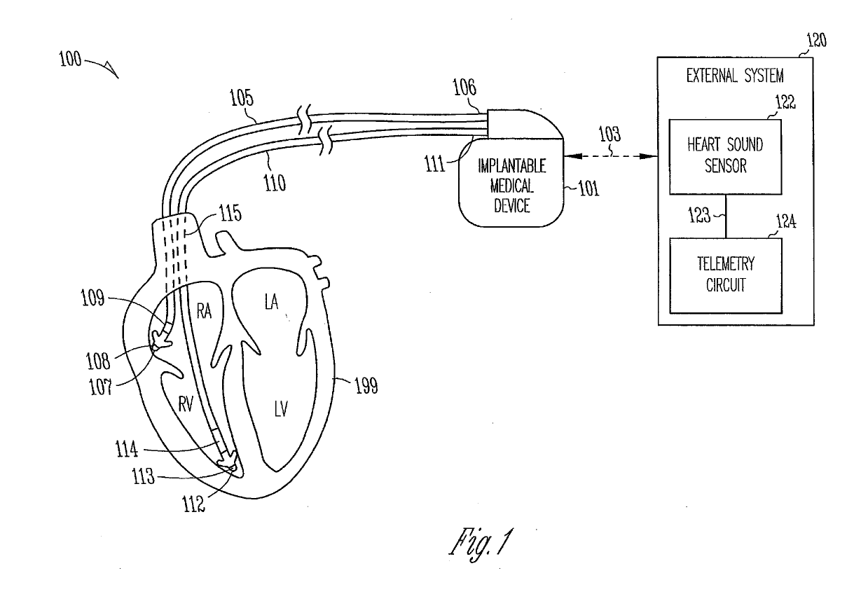

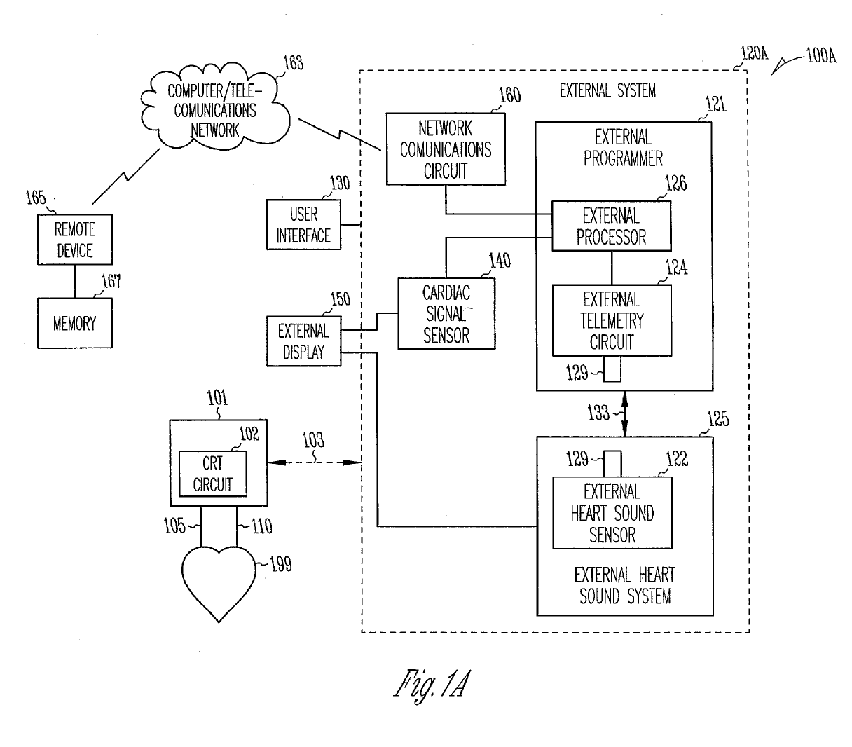

FIG. 1 is an illustration of an example of a CRM system 100 and portions of an environment in which the CRM system 100 operates. In this example, CRM system 100 includes an implantable medical device (IMD) 101 that is electrically coupled to a heart 199 through one or more electrodes, such as on leads 105 and 110. An external system 120 communicates with implantable medical device O101 via a telemetry link 103.

In certain examples, the implantable medical device 101 can include an implantable cardiac rhythm management device that can deliver one or more of pacing, cardiac resynchronization or anti-tachyarrhythmia therapies such as anti-tachyarhythmia pacing (ATP), cardioversion and defibrillation therapies. The implantable medical device 101 can include one or more of other monitoring and/or therapeutic devices such as a cardiac pacer, a cardioverter/defibrillator, a neural stimulator, a drug delivery device, and a biological therapy device. Implantable medical device 101 generally includes a hermetically sealed can housing an electronic circuit that typically senses physiological signals and/or delivers therapeutic electrical pulses. The hermetically sealed can may also function as an electrode, such as for sensing and/or pulse delivery purposes. In certain examples, as illustrated in FIG. 1, the electronic circuit senses an atrial electrogram and/or a ventricular electrogram from heart 199 and delivers pacing, cardioversion, and/or defibrillation pulses to heart 199. In this example, a lead 105 is illustrated as a pacing lead that includes a proximal end 106 connected to implantable medical device 101 and a distal end 107 placed in the right atrium (RA) of heart 199. A pacing-sensing electrode 108 (referred to as the “RA tip” electrode) is located at distal end 107. Another pacing-sensing electrode 109 (referred to as the “RA ring” electrode) is located near distal end 107. Electrodes 108 and 109 are generally electrically connected to implantable medical device 101, such as via separate conductors in lead 105, to allow sensing of the atrial electrogram and/or delivery of atrial pacing pulses. In this example, lead 110 is illustrated as a defibrillation lead that includes a proximal end 111 connected to implantable medical device 101 and a distal end 112 placed in the right ventricle (RV) of heart 199. A pacing-sensing electrode 113 (referred to as the “RV tip” electrode) is located at distal end 112. A defibrillation electrode 114 (referred to as the “RV coil” electrode) is located near distal end 112 but electrically separated from pacing-sensing electrode 113. Another defibrillation electrode 115 (referred to as the “SVC coil” electrode) is located at a distance from distal end 112, such as for placement in the superior vena cava (SVC). In certain examples, electrode 115 is electrically connected to the hermetically sealed can. Electrodes 113, 114, and 115 are electrically connected to implantable medical device 101, such as via separate conductors in lead 110. Electrode 113 allows sensing of the ventricular electrogram and/or delivery of ventricular pacing pulses. Electrodes 114 and 115 allow sensing of the ventricular electrogram and/or delivery of ventricular cardioversion and defibrillation pulses.

In this example, the external system 120 includes an external heart sound sensor 122, an external telemetry circuit 124, and a communication link 123. In at least one example, the external system 120 can be a local or remote external programmer for the implantable medical device 101. The telemetry circuit 124 generally communicates with implantable medical device 101, such as via telemetry link 103, and generally allows altering the functionality of the implantable device 101, for example, as a function of one or more sensed heart sounds. The telemetry link 123 may be a wired link or more typically a wireless link, and allows for communication between the telemetry circuit 124 and heart sound sensor 122.

The telemetry link 103 can be a wireless communication link providing for bidirectional data transmission between implantable medical device 101 and the external system 120. In certain examples, telemetry link 103 is an inductive telemetry link. In an alternative example, telemetry link 103 is a far-field radio-frequency telemetry link. Other types of a telemetry link 103 (e.g., ultrasound, infrared, etc.) could similarly be used. The external telemetry circuit 124 is adapted to communicate, through the telemetry link 103, with the implantable medical device 101. In this way, the external system 120 can automatically program at least one parameter of the implantable medical device 101, such as by using information about the at least one heart sound received from the external heart sound sensor 122. In an example in which the telemetry link 103 provides for data transmission from implantable medical device 101 to the external system 120, the transmission can include transmitting real-time physiological data acquired by implantable medical device 101, extracting physiological data acquired by and stored in implantable medical device 101, extracting therapy history data stored in implantable medical device 101, and/or extracting data indicating an operational status of implantable medical device 101 (e.g., battery status and/or lead impedance). In an example in which the telemetry link 103 provides for data transmission from the external system 120 to the implantable medical device 101, this transmission can include, for example, programming implantable medical device 101 to acquire physiological data, programming implantable medical device 101 to perform at least one self-diagnostic test (such as for a device operational status), programming implantable medical device 101 to enable an available monitoring or therapeutic function, and/or programming implantable medical device 101 to adjust one or more therapeutic parameters such as pacing, cardioversion, and/or defibrillation parameters.

FIG. 1A illustrates another example of a CRM system 100A. In this example, the CRM system 100A of FIG. 1A includes an external processor 126 within the external system 120A. The external processor 126 can be configured to automatically determine a value of a parameter of the implantable medical device 101, such as by using the heart sound information sensed by the external heart sound sensor 122. A user interface 130 can be coupled to the external system 120A. The user interface 130 can receive input from a physician or health care provider in connection with programming the IMD 101. Such input may be used to obtain physician-confirmation of a value of a parameter for the IMD 101, for example, as calculated by the external processor 126 as a function of the one or more heart sounds sensed by the external heart sound sensor 122.

In the example of FIG. 1A, the external processor 126 and the external telemetry circuit 124 are included within an external programmer 121. The external heart sound sensor 122 is associated with an external heart sound system 125. The external heart sound system 125 and the external programmer 121 can communicate via a communication link 133. FIG. 1A further illustrates an example in which the external system 120A can include an external cardiac signal sensor 140. However, the cardiac signal sensor may also be implantable. An external display 150 can be coupled to the external heart sound sensor 122 and the cardiac signal sensor 140. The external display 150 can be configured to display a heart sound signal and/or a cardiac signal. A cardiac resynchronization therapy circuit 102 can be part of the implantable medical device 101. The cardiac resynchronization circuit 102 can be used to spatially coordinate the contraction of different regions of the heart, with or without affecting the rate of such heart contractions. The cardiac resynchronization circuit 102 can have one or more of its parameters altered by the external system 120A based on the heart sounds sensed by the external heart sound sensor 122. A serial port 129 can couple the external heart sound sensor 122 and the external telemetry circuit 124. The serial port 129 can be an RS-232 port, a Universal Serial Bus (USB) port, or a fly-by-wire connection.

FIG. 1A further illustrates that the external system 120A can include a network communication circuit 160, which can communicate with a remote device 165. The communication between the communication circuit 160 and the remote device 165 can be over a computer or telecommunications network 163. In an example, a physician can receive data at the remote device 165 from the external programmer 121, such as cardiac and heart sound data. The physician can examine this data, including the changes to the parameters automatically calculated by the processor 126, and send a confirmation or a rejection from the remote device 165, over the network 163, to the external system 120A. The remote device 165 can include a memory 167. The memory 167 can serve as a central repository for data that is received for multiple IMDs 101, such as for multiple patients in the care of a physician.

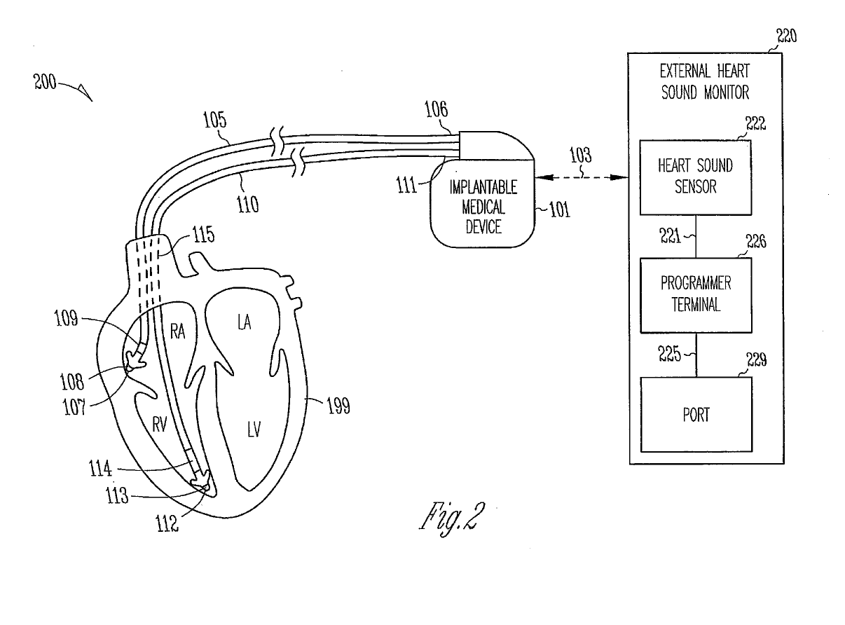

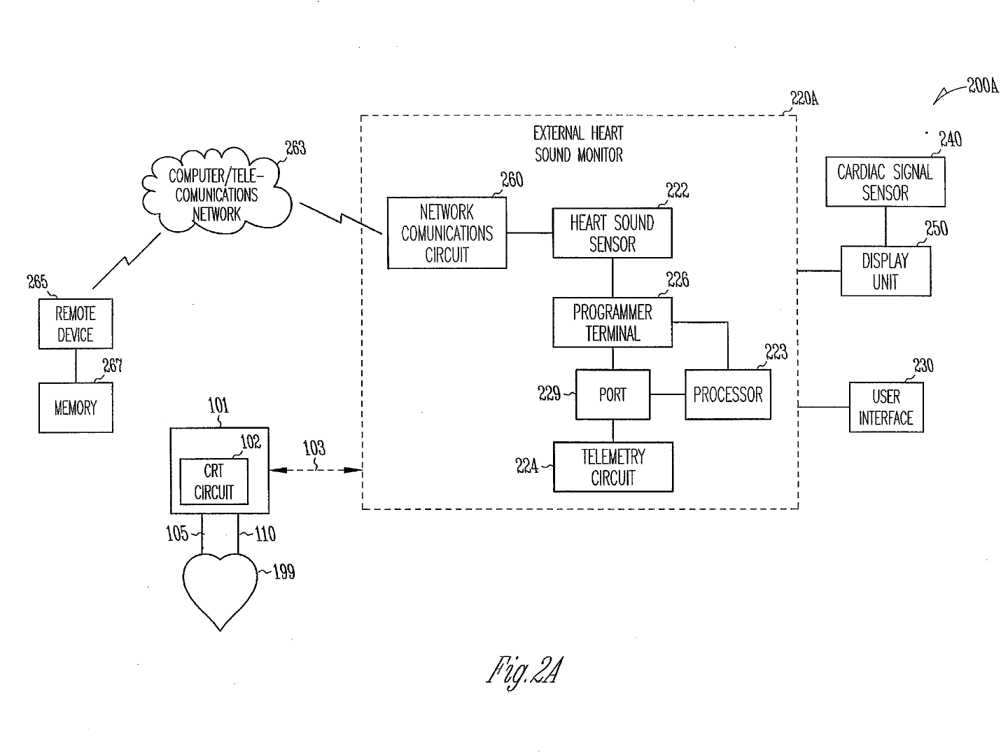

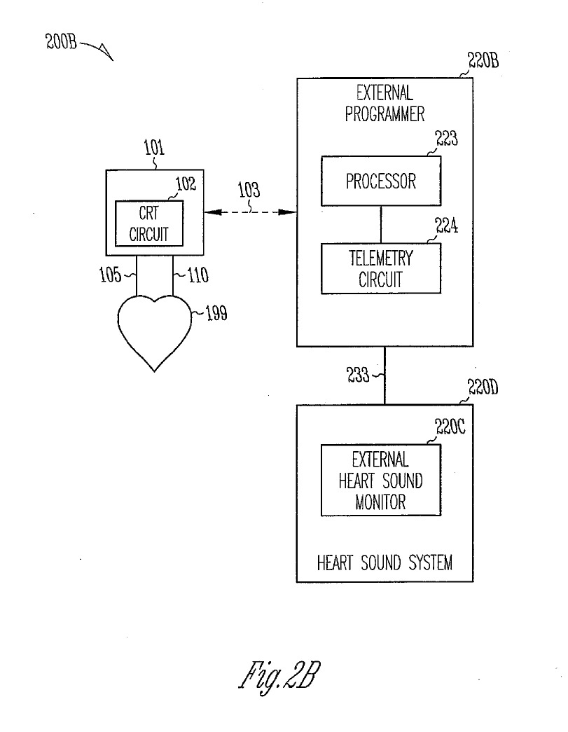

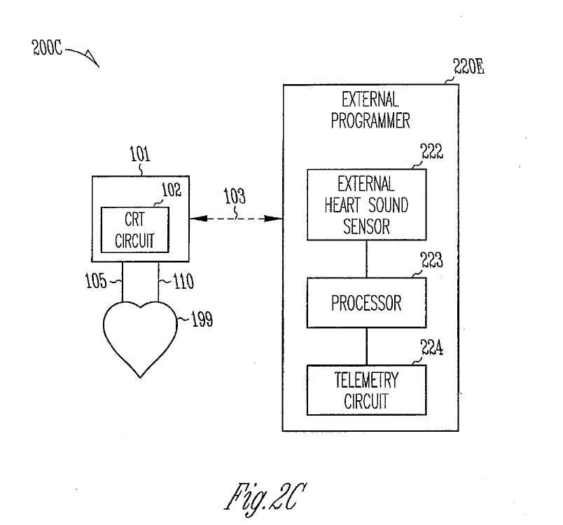

FIG. 2 is an illustration of an example of a CRM system 200 and portions of the environment in which the CRM system 200 operates. CRM system 200 is another example of CRM system 100 and includes implantable medical device 101 that is electrically coupled to a heart 199 through one or more electrodes, such as on leads 105 and 110. An external heart sound monitor 220 communicates with implantable medical device 101 via a telemetry link 103.

External heart sound monitor 220 is another example of external system 120. In this example, the external heart sound monitor 220 includes programmer/terminal 226, a heart sound sensor 222, a port 229, a communication link 221, and a communication link 225. In an example, the port 229 automatically programs at least one parameter of the IMD 101 using a wireless communication circuit and information from at least one heart sound of the external heart sound sensor 222. The port 229 can include a serial RS-232 port, a Universal Serial Bus (USB) port, or a fly-by-wire connection.

FIG. 2A illustrates one or more other features that may be part of the CRM system 200A. A display unit 250 may be connected to the external heart sound monitor 220A. The display unit 250 can display a heart sound signal received from the heart sound sensor 222. The display of the heart sound signal generally permits a physician or other health care provider to use the heart sound signal, such as in the diagnosis and/or treatment of a patient. The display unit 250 may also have coupled to it an external or implantable cardiac signal sensor 240. The display unit 250 can also display a cardiac signal from the implantable cardiac signal sensor 240. A physician or other health care provider can then examine one or more cardiac signals on the display unit 250. The system may also concurrently display one or more cardiac signals and one or more heart sound signals on the display unit. This permits a physician to examine the relationship between a heart sound signal and a cardiac signal.

FIG. 2A illustrates an example in which the external heart sound monitor 220A may further include a telemetry circuit 224 coupled to the port 229. In this example of the external heart sound monitor 220A, the external telemetry circuit 224 can be configured to communicate with the IMD 101, such as to automatically program a parameter of the IMD 101 using heart sound information received from the external heart sound sensor 222. For example, the external telemetry circuit 224 can be configured to communicate with the IMD 101 to automatically adjust an atrioventricular (AV) delay, an interventricular (VV) delay, an LV offset, an intraventricular delay, a selected electrode for delivering an electrostimulation pulse, and/or other cardiac resynchronization therapy (CRT) parameter. For example, the external telemetry circuit 224 may be configured to communicate with the IMD 101 to automatically adjust a cardiac resynchronization therapy (CRT) parameter that controls a cardiac resynchronization therapy circuit 102. The CRT parameter can be adjusted in such a way so as to decrease or minimize an observed heart sound amplitude received from the external heart sound sensor 222. Such heart sounds can include an S1, S2, S3, and/or S4 heart sounds.

The external heart sound monitor 220A may further include a processor 223 coupled to the terminal/programmer 226 and the port 229. The processor 223 can be configured to automatically determine a suggested value of a therapy control parameter of the IMD 101, such as by using information about a heart sound received from the external heart sound sensor 222. In another example, illustrated in FIG. 2B, a CRM system 200B includes the external processor 223 and the external telemetry circuit 224 housed within an external programmer 220B for the IMD 101. In this example, the external heart sound monitor 220C is associated with an external heart sound system 220D that is housed separately from the external programmer 220B. The external heart sound system 220D and the external programmer 220B can be coupled together through a wired or wireless communication link 233. In this example, the external heart sound system 220D can provide heart sound information to the external processor 223 housed in the external programmer 220B of the CRM system 200B. In this way, the external processor 223 can use such heart sound information to adjust one or more CRT or other therapy control parameters in response to such heart sound information, such as to reduce or minimize one or more heart sound amplitudes.

FIG. 2C illustrates another example of a CRM system 200C in which the external processor 223, the external telemetry circuit 224, and the external heart sound sensor 222 are all part of a remote or local external programmer 220E for programming the IMD 101. In this example, the external heart sound sensor 222 need not be part of a separately housed external heart sound monitor. In this example, the external heart sound sensor 222 can provide heart sound information to the external processor 223 of the external programmer 220E, which can then use such heart sound information to program one or more CRT or other therapy control parameters of the IMD 101, such as to reduce or minimize one or more heart sound amplitudes.

FIG. 2A further illustrates an example in which the system 200A can include an external network communication circuit 260. The network communication circuit 260 can communicate with a remote device 265, such as through a wired or wireless computer and/or telecommunications network 263. The remote device 265 may further include a memory 267. This memory can serve as a central memory or repository for heart sound information, IMD parameter settings, or other data that can be received from various IMDs 101 that are respectively associated with a multitude of corresponding patients.

FIG. 2A further shows an example in which the system 200A can include a user interface 230. In this example, through the user interface, a physician or other health care provider may confirm or reject a value of a CRT or other therapy control parameter that was automatically calculated by the processor 223 based on heart sound information sensed by the heart sound sensor 222.

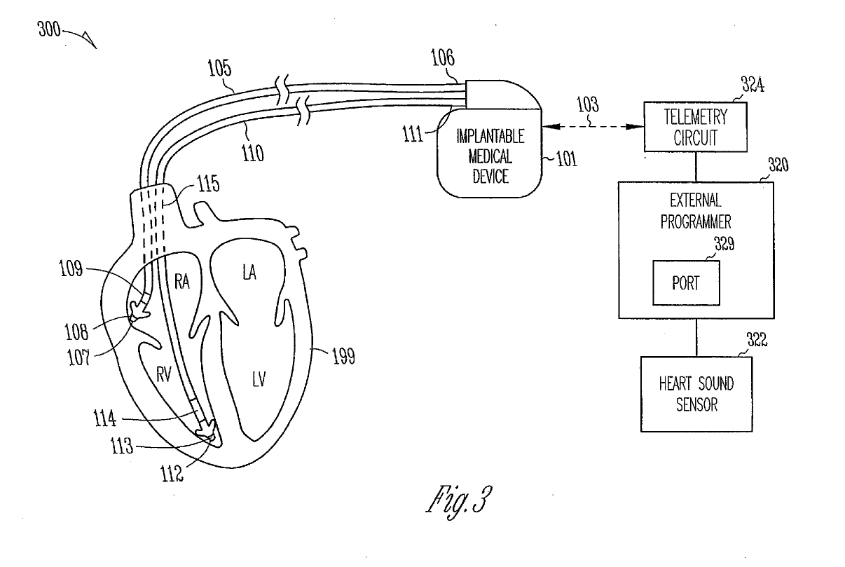

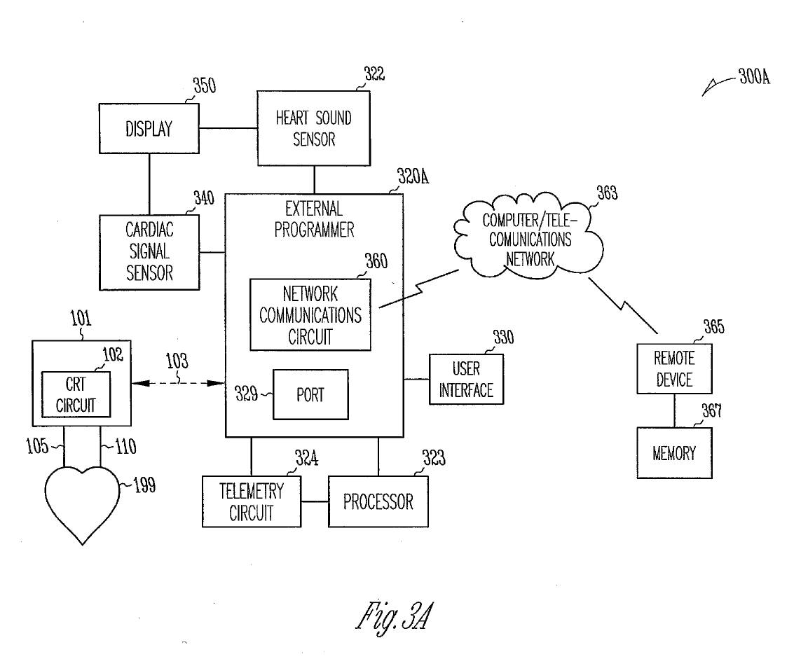

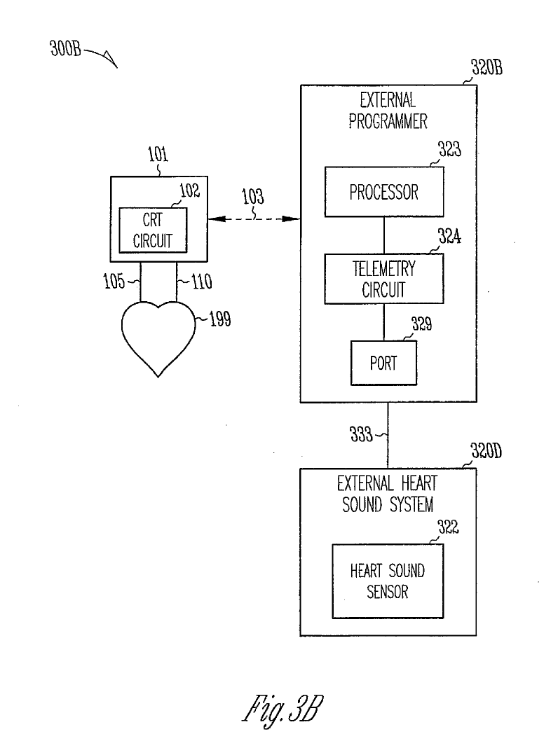

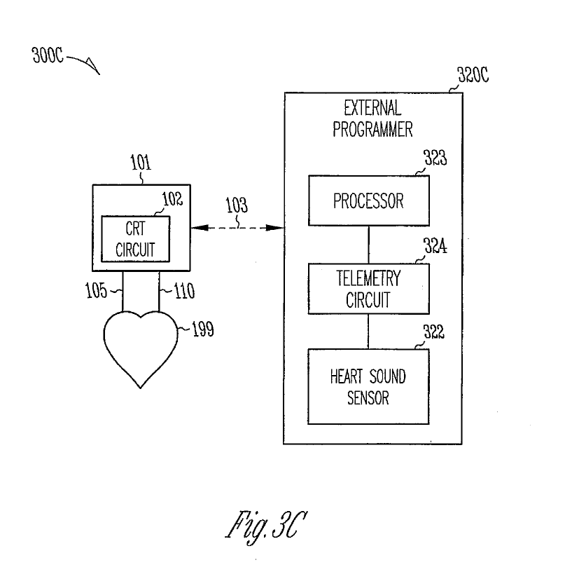

FIG. 3 is an illustration of an example of a CRM system 300 and portions of the environment in which CRM system 300 operates. CRM system 300 is another example of CRM system 100 and includes implantable medical device 101 that is electrically coupled to a heart 199 through one or more electrodes, such as on leads 105 and 110. An external programmer 320 through a telemetry circuit 324 communicates with the implantable medical device 101 via a telemetry link 103.

External programmer 320 is another example of external system 120 and includes a port 329. In this example, an external heart sound sensor 322 is coupled to the external programmer 320 through the port 329. In this example, an external telemetry circuit 324 is also coupled to or included in the external programmer 320. The telemetry circuit 324 can be configured to communicate with the IMD 101 and to automatically program at least one parameter of the IMD 101, such as using heart sound information received form the external heart sound sensor 322. The port 329 can include a serial RS-232 port, a Universal Serial Bus (USB) port, or a fly-by-wire connection.

FIG. 3A illustrates another example of the CPM system 300A. In this example, the CRM system 300A of FIG. 3A includes an external processor 323. The processor 323 is coupled to the external programmer 320A and the external telemetry circuit 324. The external processor 323 can be configured to automatically determine a value of a parameter of the IMD 101, such as by using heart sound information received from the heart sound sensor 322 via the external programmer 320A. A user interface 330 is connected to or included in the external programmer 320A. The user interface 330 can be used by a physician or other health care provider to confirm or reject one or more parameters of the IMD 101 calculated by the processor 323 based on heart sounds received from the external heart sound sensor 322.

The external processor 323 and the external telemetry circuit 324 can be included within an external programmer 320B as shown in the CRM system 300B of FIG. 3B. Additionally or alternatively, the external heart sound sensor 322 can be associated with an external heart sound system 320D. The external heart sound system 320D in this example is housed separately from the external programmer 320B. In this example, the external heart sound system 320D and the external programmer 320B are configured such that they can be connected together by one or more wires, or such that they can be wirelessly coupled together such as by wireless link 333. In another example, as shown in FIG. 3C, a CRM system 300C includes an external programmer 320C that houses the external processor 323, the external telemetry circuit 324, and the external heart sound sensor 322 in an external programmer 320C. In either such example, the heart sound information can be used to automatically program one or more CRT or other therapy control parameters, such as to reduce or minimize one or more heart sounds.

FIG. 3A further illustrates that the CRM system 300A can include an implantable and/or external cardiac signal sensor 340. The cardiac signal sensor 340 and/or the external heart sound sensor 322 can be coupled to an external display 350. The external display 350 displays one or more heart sound signals and/or one or more cardiac signals, which a physician may use to examine and/or diagnose a patient.

The external telemetry circuit 324 can be configured to perform one or more of several functions. For example, the telemetry circuit 324 may be configured to communicate with the IMD 101 to automatically adjust a cardiac resynchronization therapy parameter in a cardiac resynchronization therapy circuit 102. This adjustment can be made in response to feedback received from the external heart sound sensor 322, such as to decrease or minimize an observed heart sound amplitude, energy, or other indicator received from the external heart sound sensor 322 via the external programmer 320, 320A, 320B or 320C. In certain examples, the adjustment of the cardiac resynchronization parameter is meant to decrease or minimize an observed S3 heart sound amplitude received from the external heart sound sensor 322 via the external programmer 320. The telemetry circuit 324 can be configured to communicate with the IMD 101, for example, to automatically adjust an atrioventricular (AV) delay, an interventricular (VV) delay, an LV offset, an intraventricular delay, and a selected electrode for delivering an electrostimulation pulse.

FIG. 3A further illustrates an example in which the external programmer system 320A can include an external network communication circuit 360. The network communication circuit 360 can communicate with a remote device 365 through a computer or telecommunications network 363. The remote device 365 may further include a memory 367. This memory can serve as a central memory or repository for data that is received from a multitude of IMDs 101 that are associated with a multitude of patients.

FIG. 3A further shows that the system 300A can include a local or remote user interface 330. Through the user interface, a physician or other health care provider may confirm or reject a value of a parameter in conjunction with the automatic programming of the parameter by the processor 323.

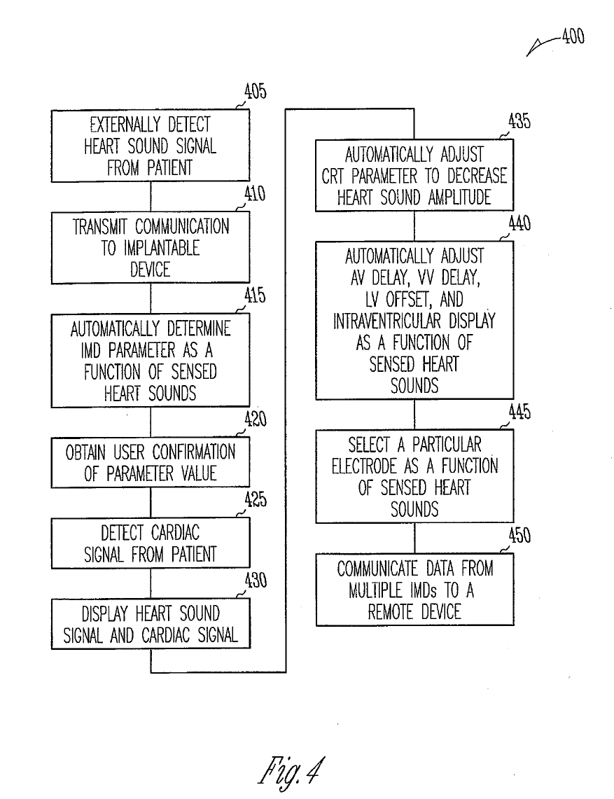

FIG. 4 illustrates an example of a process 400 to externally sense one or more heart sounds and to use information from those sensed heart sounds to automatically program an implantable medical device. At 405, a heart sound signal can be externally detected from a patient. At 410, a communication can be transmitted to the implantable medical device. The communication can include automatically programming at least one CRT or other therapy control parameter of the implantable medical device using the feedback information about the at least one heart sound. At 415, a value of the at least one parameter of the implantable medical device can be automatically determined using the feedback information about the at least one heart sound received from the external heart sound sensor. At 420, a user-confirmation of the automatically-determined value of the at least one parameter can be obtained. At 425, a cardiac signal can be detected from the patient, and at 430 the heart sound signal and the cardiac signal can be displayed on an external display unit. At 435, a cardiac resynchronization therapy or other therapy control parameter can be automatically adjusted, such as to decrease or minimize an externally detected heart sound amplitude or other indicator derived from one or more heart sounds over one or more cardiac cycles. This automatic adjustment can be used to tend to decrease or minimize an externally detected S3 heart sound amplitude. At 440, for example, at least one of an atrioventricular (AV) delay, an interventricular (VV) delay, an LV offset, and an intraventricular delay can be automatically adjusted, such as a function of one or more of the externally sensed heart sounds. At 445, for example, a particular electrode of the implantable medical device can be selected for delivering at least one electrostimulation pulse based on the one or more externally sensed heart sounds. At 450, data from multiple implantable medical devices is communicated to a remote device, such as by using a computer or telecommunications network. The acts of the process illustrated in FIG. 4 depict only one particular example; not all of these acts need be performed in any particular example of a process to use one or more externally sensed heart sounds to automatically adjust one or more CRT or other therapy control parameters for an implantable medical device.

One or more examples of the present disclosure may be used in conjunction with other medical equipment in the market such as the AUDICOR® systems of Inovise Medical, Inc. of Portland, Oreg.

It is to be understood that the above detailed description is intended to be illustrative, and not restrictive. Other embodiments will be apparent to those of skill in the art upon reading and understanding the above description. The scope of the invention should, therefore, be determined with reference to the appended claims, along with the full scope of equivalents to which such claims are entitled.

In the above detailed description of embodiments of the disclosure, various features are grouped together in one or more embodiments for streamlining the disclosure. This is not to be interpreted as reflecting an intention that the claimed embodiments of the invention require more features than are expressly recited in each claim. Rather, as the following claims reflect, inventive subject matter lies in less than all features of a single disclosed embodiment. Thus the following claims are hereby incorporated into the detailed description of embodiments, with each claim standing on its own as a separate embodiment. It is understood that the above description is intended to be illustrative, and not restrictive. It is intended to cover all alternatives, modifications and equivalents as may be included within the scope of the disclosure as defined in the appended claims. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein,” respectively. Moreover, the terms “first,” “second,” and “third,” etc., are used merely as labels, and are not intended to impose numerical requirements on their objects.

As used in this disclosure, the term “circuit” is broadly meant to refer to hardware, software, and a combination of hardware and software. That is, a particular function may be implemented in specialized circuits, in software executing on general processor circuits, and/or a combination of specialized circuits, generalized circuits, and software.

The abstract is provided to comply with 37 C.F.R. 1.72(b) to allow a reader to quickly ascertain the nature and gist of the technical disclosure. The Abstract is submitted with the understanding that it will not be used to interpret or limit the scope or meaning of the claims.

1. A system comprising:
an external heart sound sensor adapted to externally detect at least one heart sound from a patient; and
an external telemetry circuit, coupled to the external heart sound sensor to receive information about the at least one heart sound, the external telemetry circuit adapted to communicate with an implantable medical device for automatically programming at least one parameter of the implantable medical device using information about the at least one heart sound received from the external heart sound sensor.

2. The system of claim 1, further comprising:
an external processor, coupled to each of the external heart sound sensor and the external telemetry circuit, the external processor configured to automatically determine a value of the at least one parameter of the implantable medical device using the information about the at least one heart sound received from the external heart sound sensor; and
a user interface to obtain user-confirmation of the value of the at least one parameter in conjunction with the automatic programming of the at least one parameter.

3. The system of claim 2, in which the external processor and the external telemetry circuit are included within an external programmer for an implantable cardiac function management device, and in which the external heart sound sensor is associated with an external heart sound system that is housed separately from the external programmer, and wherein the external heart sound system and the external programmer are adapted to be communicatively intercoupled.

4. The system of claim 2, in which at least a portion of the external processor, the external telemetry circuit, and the external heart sound sensor are all included within an external programmer for an implantable cardiac function management device.

5. The system of claim 1, further comprising:
an implantable or external cardiac signal sensor; and
an external display, coupled to the external heart sound sensor and the implantable or external cardiac signal sensor, the external display configured to display a heart sound signal and a cardiac signal;
in which the external telemetry circuit is adapted to communicate with the implantable medical device for automatically adjusting a cardiac resynchronization therapy parameter to decrease or minimize an observed heart sound amplitude received from the external heart sound sensor; and
in which the observed heart sound amplitude includes an observed S3 heart sound amplitude received from the external heart sound sensor.

6. The system of claim 1, further comprising the implantable medical device; and
in which the external telemetry circuit is adapted to communicate with an implantable medical device for automatically adjusting at least one of an atrioventricular (AV) delay, an interventricular (VV) delay, an LV offset, an intraventricular delay, and a selected electrode for delivering an electrostimulation pulse; and
in which the implantable medical device includes a cardiac resynchronization therapy circuit.

7. The system of claim 1, further comprising:
a remote device; and
an external network communication circuit adapted to communicate with the remote device using a computer or telecommunications network;
in which the remote device includes a centralized repository for data received from multiple implantable medical devices.

8. The system of claim 1, further comprising one or more of a serial port, parallel port, and a wireless port, coupled between the external heart sound sensor and the external telemetry circuit; and
in which the serial port includes an RS-232 port, a Universal Serial Bus (USB) port, or a fly-by-wire port.

9. A method comprising:
externally detecting at least one heart sound signal from a patient; and
communicating with an implantable medical device, the communicating including automatically programming at least one parameter of the implantable medical device using information about the at least one heart sound.

10. The method of claim 9, further comprising:
automatically determining a value of the at least one parameter of the implantable medical device using the information about the at least one heart sound received from the external heart sound sensor;
obtaining user-confirmation of the value of the at least one parameter in conjunction with the automatic programming of the at least one parameter; and
detecting a cardiac signal from the patient, and displaying the heart sound signal and the cardiac signal on an external display unit.

11. The method of claim 9, further comprising:
automatically adjusting a cardiac resynchronization therapy parameter to decrease or minimize an externally detected heart sound amplitude;
in which the externally detected heart sound amplitude includes an externally detected S3 heart sound amplitude.

12. The method of claim 9, further comprising automatically communicating with the implantable medical device to automatically adjust at least one of an atrioventricular (AV) delay, an interventricular (VV) delay, an LV offset, an intraventricular delay, and a selected electrode for delivering an electrostimulation pulse.

13. The method of claim 9, further comprising communicating data from multiple implantable medical devices to a remote device using a computer or telecommunications network.

14. A system comprising:
an external heart sound monitor comprising:
a terminal configured to receive at least one heart sound signal from an external heart sound sensor;
a port configured to communicate with an implantable medical device for automatically programming at least one parameter of the implantable medical device using information about the at least one heart sound received from the external heart sound sensor.

15. The system of claim 14, further comprising:
a display unit to display the at least one heart sound signal received from the external heart sound sensor; and
an external or implantable cardiac signal sensor coupled to the display, the external display configured to display a heart sound signal and a cardiac signal.

16. The system of claim 14, further comprising:
an external telemetry circuit, coupled to the port, the external telemetry circuit adapted to communicate with the implantable medical device for the automatic programming of the at least one parameter of the implantable medical device using information about the at least one heart sound received from the external heart sound sensor; and
an external processor, coupled to each of the terminal and the port, the external processor configured to automatically determine a value of the at least one parameter of the implantable medical device using the information about the at least one heart sound received from the external heart sound sensor;
in which the external telemetry circuit is adapted to communicate with the implantable medical device for automatically adjusting at least one of an atrioventricular (AV) delay, an interventircular (VV) delay, an LV offset, an intraventricular delay, and a selected electrode for delivering an electrostimulation pulse;
in which the external processor and the external telemetry circuit are included within an external programmer for an implantable cardiac function management device, and in which the external heart sound monitor is associated with an external heart sound system that is housed separately from the external programmer, and wherein the heart sound system and the external programmer are adapted to be communicatively intercoupled; and
in which at least a portion of the external processor, the external telemetry circuit, and the external heart sound sensor are all included within an external programmer for an implantable cardiac function management device.

17. The system of claim 16,
in which the external telemetry circuit is adapted to communicate with the implantable medical device for automatically adjusting a cardiac resynchronization therapy parameter to decrease or minimize an observed heart sound amplitude received from the external heart sound sensor; and
in which the external telemetry circuit is adapted to communicate with the implantable medical device for automatically adjusting a cardiac resynchronization therapy parameter to decrease or minimize an observed S3 heart sound amplitude from the external heart sound sensor.

18. The system of claim 14, further comprising the implantable medical device; wherein the implantable medical device includes a cardiac resynchronization therapy circuit.

19. The system of claim 14, further comprising:
a remote device; and
an external network communication circuit adapted to communicate with the remote device using a computer or telecommunications network;
in which the remote device includes a central repository for data received from multiple implantable medical devices.

20. The system of claim 14, further comprising a user interface to obtain user-confirmation of the value of the at least one parameter in conjunction with the automatic programming of the at least one parameter;
wherein the port includes a serial RS-232 port, a Universal Serial Bus (USB) port, or a fly-by-wire port.

21. A system comprising:
an external programmer comprising a port configured to receive at least one heart sound from an external heart sound sensor; and
an external telemetry circuit, coupled to the external programmer, the external telemetry circuit adapted to communicate with an implantable medical device for automatically programming at least one parameter of the implantable medical device using information about the at least one heart sound received from the external programmer.

22. The system of claim 21, further comprising:
an external processor, coupled to each of the external programmer and the external telemetry circuit, the external processor configured to automatically determine a value of the at least one parameter of the implantable medical device using the information about the at least one heart sound received from the external programmer; and
a user interface to obtain user-confirmation of the value of the at least one parameter in conjunction with the automatic programming of the at least one parameter;
in which the external processor and the external telemetry circuit are included within the external programmer, and in which the external heart sound sensor is associated with an external heart sound system that is housed separately from the external programmer, and wherein the external heart sound system and the external programmer are adapted to be communicatively intercoupled; and
in which at least a portion of the external processor, the external telemetry circuit, and the external heart sound sensor are all included within the external programmer.

23. The system of claim 21, further comprising:
an implantable or external cardiac signal sensor; and
an external display, coupled to the external heart sound sensor and the implantable or external cardiac signal sensor, the external display configured to display a heart sound signal and a cardiac signal;
in which the external telemetry circuit is adapted to communicate with the implantable medical device for automatically adjusting a cardiac resynchronization therapy parameter to decrease or minimize an observed heart sound amplitude received from the external programmer; and
in which the external telemetry circuit is adapted to communicate with the implantable medical device for automatically adjusting a cardiac resynchronization therapy parameter to decrease or minimize an observed S3 heart sound amplitude received from the external programmer.

24. The system of claim 21, further comprising:
a remote device;
an external network communication circuit adapted to communicate with the remote device using a computer or telecommunications network; and
the implantable medical device;
in which the external telemetry circuit is adapted to communicate with an implantable medical device for automatically adjusting at least one of an atrioventricular (AV) delay, an interventricular (VV) delay, an LV offset, an intraventricular delay, and a selected electrode for delivering an electrostimulation pulse; and
in which the implantable medical device includes a cardiac resynchronization therapy circuit; and
in which the remote device includes a centralized repository for data received from multiple implantable medical devices.

25. The system of claim 21, comprising a serial port coupled between the external heart sound sensor and the external telemetry circuit; in which the serial port includes an RS-232 port, a Universal Serial Bus (USB) port, or a fly-by-wire port.
